(19) United States
(12) Patent Application Publication
(10) Pub. No.: US 2025/0144033 A1
(43) Pub. Date: May 8, 2025

(54) METHOD OF MANUFACTURING MULTIPLE DOSAGE FORMS WITH PERSONALISED COMPOSITION AND DEVICE FOR CARRYING OUT THIS METHOD

(71) Applicant: VYSOKA SKOLA CHEMICKO-TECHNOLOGICKA V PRAZE, Praha 6 – Dejvice (CZ)

(72) Inventors: Erik SONNTAG, Cervena Voda (CZ); Frantisek STEPANEK, Praha 4 (CZ); Jan VRBA, Praha 10 (CZ); Elizaveta MUTYLO, Praha 5 - Stodulky (CZ); Silvia SLEZAKOVA, Bratislava (SK)

(21) Appl. No.: 18/837,386

(22) PCT Filed: Feb. 3, 2023
(86) PCT No.: PCT/CZ2023/050006

§ 371 Date: Aug. 9, 2024

(30) Foreign Application Priority Data
Feb. 11, 2022 (CZ) PV 2022-66

(51) Int. Cl. A61K 9/48; G05B 19/418

(52) U.S. Cl. CPC A61K 9/4833; G05B 19/4189; G05B 2219/31276; G05B 2219/32368

(57) ABSTRACT

The present invention relates to a computer-implemented method for preparing multiple dosage forms with personalised composition, comprising the following steps:
(i) a control unit generates an input protocol of a personalised composition of a multiple dosage form, containing information about the type and amount of individual active substances, the type of cartridge, and the type and number of capsules;
(ii) a cartridge of the required type according to the input protocol, labelled with a specific machine-readable code, is placed in the automatic transport system;
(iii) at least two different types of sub-units are placed into at least two dosing stations, the sub-unit having sizes in the range of from 0.1 to 4 mm, and containing a different active ingredient and/or different amounts of the same active ingredient and/or having coatings with different solubility and/or possibly containing different pharmaceutical excipients according to the input protocol;
(iv) a pre-defined number of capsule bodies is placed in the cartridge placed in an automatic transport system;
(v) the automatic transport system transports the cartridge with capsules to the first dosing station, which doses a pre-defined number of the first sub-units into the capsule bodies placed in the cartridge;
(vi) the automatic transport system transports the cartridge with capsules to the next dosing station, which doses a pre-defined number of the next sub-units into the capsule bodies placed in the cartridge; and this step is repeated according to the input protocol until the capsules are filled with all of the pre-defined types of sub-units;
(vii) the capsules are closed with caps;
(viii) the quality control of capsules and discarding of the non-compliant ones;
(ix) packaging and labelling with a batch number.

The present invention further relates to a device for carrying out this method.

### FIELD OF ART

The present invention relates to a technology for the automated manufacturing of multiple dosage forms with a personalised composition and release profile of active ingredients. Multiple dosage forms herein refer to pharmaceutical formulations in the form of capsules containing several types of sub-units, differing in their active ingredient and/or different concentrations thereof and, where appropriate, in the composition and/or amount of pharmaceutical excipients. Thus, the invention allows dispensing a predefined amount of at least one active ingredient with a predefined release profile into capsules according to the patient's individual needs. The invention further relates to a device for carrying out this method and to dosage forms preparable by this method.

### BACKGROUND ART

Surveys of medication prescribing trends showed that in the population sample studied, approximately 10 percent of patients overall and more than 30 percent of patients aged 65 years and over were taking five or more different medications every day. This phenomenon is referred to as polypharmacy. Furthermore, the studies mentioned above showed that in the group of patients who took a combination of three different medications, 98 percent of the medication regimens (i.e. the specific combination of active ingredients and their strengths) were unique, i.e. only 2 percent of the patients in the study sample were prescribed the same medication at the same strength. In the case of patients with five or more different medications, the rate of the uniqueness of medication regimens was even 100 percent, i.e. each patient in the study sample took a different combination of prescribed medications and strengths.

The complexity of the medication regimen caused by the need to take many different medications often leads to reduced patient adherence to treatment. This results in reduced treatment efficacy and severe health complications due to underdosing or overdosing and the associated costs of subsequent treatment or hospitalisation.

Adherence to prescribed treatment is reduced mainly in patients who have to take combinations of multiple different medications at specific times of the day. In addition, patients often have to titrate the dose, i.e. take more than one tablet of a given medicine, take medicine only every other day, or split the tablets in half or otherwise. The available data show that older age is a negative factor for medical adherence to the medication regimen due to reduced motor and memory abilities.

Polypharmacy and/or the need to titrate the dose occur in a number of disease groups such as cardiovascular diseases, hypertension, type 2 diabetes, bleeding disorders, hormone regulation disorders, chronic pain, chemotherapy for cancer treatment, HIV, patients with immune disorders, liver diseases, chronic inflammation, lung diseases, parasitic diseases, post-surgical conditions (e.g. post-transplant), patients with attention deficit hyperactivity disorder (ADHD), and others.

For example, patients clinically diagnosed with ADHD are typically treated with a combination of psychopharmaceuticals, with treatment being patient-specific and requiring precise dose titration of the different active ingredients. Optimisation of the medication regimen is done by prescribing small doses of individual medications with different release profiles to be taken by the patient at a given time of day. The prescription is then adjusted based on the response to the medication. However, it turns out that adherence to a strict medication regimen is very problematic for many patients.

Similarly, in the case of tuberculosis (TB), it has been shown that combining multiple medications is not only more effective than single-medication therapy but also reduces the risk of resistance. However, even in the case of TB, especially in children, careful dose titration and adherence to the prescribed regimen are necessary, which can be a problem, especially in developing countries. Furthermore, the benefit of combination therapy is well-documented in cardiovascular diseases.

One proven way to improve patient adherence to medication is to reduce the number of different dosage forms (tablets, capsules, etc.) the patient must take simultaneously. For this reason, fixed-dose combinations (FDCs), in which several active ingredients are combined into one dosage form, are now used. However, for the development, registration and industrial production of FDCs to make technological and economic sense, the specific combination of active ingredients and strengths must be suitable for a sufficiently large cohort of patients. A single production batch in industrial pharmaceutical manufacturing can represent several hundred thousand to several million tablets. Therefore, FDCs are currently produced only for the most commonly encountered medication combinations in a few of the most commonly prescribed strength combinations. An example of a FDC on the market is Caramlo® (Zentiva) containing the active ingredients candesartan and amlodipine in 16/10 mg and 8/5 mg strengths. Other examples of FDCs include Twynsta® (Boehringer-Ingelheim) containing the active ingredients valsartan and amlodipine in 40/5 mg, 40/10 mg, 80/5 mg and 80/10 mg strengths, or Norvasc Protect® (Pfizer) containing amlodipine and atorvastatin in 5/10 mg, 5/20 mg, 10/10 mg and 10/20 mg strengths.

A significant limitation and disadvantage of FDCs is the inability to easily titrate the dose and their impracticality for a larger number of active ingredients, both for technical reasons (multilayer tablets can be reliably compressed on an industrial scale for a maximum of two to three layers) and economic reasons (the number of different combinations increases exponentially for multiple active ingredients and the size of the patient group prescribed the same combination of active ingredients and strengths decreases proportionally).

For example, for the triplet of candesartan (4 mg, 8 mg, 16 mg, 32 mg), amlodipine (2.5 mg, 5 mg, 10 mg) and hydrochlorothiazide (12.5 mg, 25 mg, 50 mg, 100 mg), which are often prescribed together in the treatment of cardiovascular diseases, the number of combinations of strengths already counts 48 options. With the increasing uniqueness of the combination of active ingredients and strengths, the industrial production of FDCs no longer makes technological and economic sense.

However, for individual patients, the existence of a simple dosage form containing all active ingredients used by the patient in the required strengths would be of significant benefit and would have the potential to improve adherence to the medication regimen. Although the possibility of ‘tailor-made’ medications containing active ingredients mixed specifically for a particular patient based on a prescription (pharmaceutical compounding) has historically existed in the field of pharmacy, the disadvantage is the considerable labour and time-consuming nature of this process, as it involves manual compounding and preparation in pharmacies. In addition, active ingredients with different release rates or pH-dependent release requirements (e.g. enterosolvent systems) cannot generally be combined in this way.

The state of the art still lacks sufficiently fast, accurate and automated technology for the manufacturing of individualised dosage forms with combinations of multiple active ingredients and the possibility of dose titration for individual patients. Its existence would be a major asset in the field of combination therapy. Physicians often do not prescribe fixed-dose combinations (FDCs) precisely because of the inability to adjust the dose of each active ingredient independently, for example, according to the blood pressure and lipid spectrum of a particular patient.

Nowadays, there are only a few technologies on the market for semi-automatic or automatic mixing of combined pharmaceutical formulations. Fully automated solutions are mainly available for liquid dosage forms where peristaltic pump compounding is used. Other patents include Hewlett-Packard's inkjet printer-based technology for spraying liquid active ingredients onto an edible substrate or 3D printing technology for the deposition of active ingredients to prepare layered tablets (U.S. Pat. Nos. 6,962,715, 7,727,576 and 7,707,964). However, the 3D printing of layered tablets, wherein different active ingredients are combined, is more suitable for preparing experimental prototypes but is not ideal for industrial production due to the low production speed and is further limited by the printability of real pharmaceutical substances. In the case of combining liquid active ingredients, mixing at the molecular level occurs, which can lead to undesirable interactions in terms of the stability of the individual active ingredients. A common drawback of both these techniques is the loss of control (inability to influence) over the dissolution profiles of individual active ingredients, leading to difficulty in achieving bioequivalence.

The possibility to prepare combination medications in solid form is offered by the semi-automatic powder mixing technology of TAILORPILL TECH LCC (WO 2012/087492), which is an extension of the previous ‘Xcelodose’ technology of Capsugel (Bryant, S. et al., Advances in powder-dosing technology. 2002: p. 95-100). Xcelodose is a technology developed not for personalised treatment but for micro-dosing powder materials into hard gelatine capsules. This method is mainly used to prepare prototypes in the first and second phases of clinical trials. The principle of the method is a technique of controlled tapping on a storage container with a perforated bottom. A small amount of powder falls through the perforated bottom into the capsule, the weight of which is controlled online by a microbalance. The control unit adjusts the intensity of the taps based on the weight information of the dosed substance in the capsule.

Similar devices, also based on gravimetrically controlled micro-dosing of small quantities of powder, are the ‘Quantos’ systems from Mettler-Toledo and the ‘Powdernium’ from Symyx. These dosing devices are primarily intended for automatic laboratory weighing of powdered reactants and analytes. Due to the principle of dosing, their use is limited to ingredients in the form of fine powder or granular materials with particle sizes in the order of units or lower tens of micrometres, where the continuous divisibility of these mixtures is used. However, for larger particles that need to be dosed discretely (per a defined number of pieces), the accuracy drops dramatically when using this dosing principle.

The aforementioned technologies do not allow precise and automatic combining of larger solid dosage forms (characteristic size from 0.1 to 4 mm), such as mini-tablets, pellets, granules or liquid marbles (collectively referred to as sub-units). Due to the size of the sub-units, the dosing accuracy by tapping on the container with a perforated bottom and by gravimetric control of the dispensed contents (thus by the mechanism used for automatic mixing of solid dosage forms in the current state of the art) is very low and it is not possible to dose/combine a precisely defined number of particles. From a formulation point of view, however, these larger particles in personalised combination therapy offer a great many advantages because, unlike powders of the active ingredient itself, they offer great formulation flexibility. In fact, they can be formulated to meet the condition of bioequivalence, i.e. so that the desired profile of the rate of release of the active ingredient can be achieved by an appropriate choice of excipients, microstructure, particle size and shape, including possible coating.

When the sub-units described above are used in combined dosage forms, the technology used to produce these dosage forms must be capable of combining and counting these particles with unit accuracy, which is not the case with the systems described above. Thus, the technology for quick, precise, automated mixing of larger pharmaceutical particles (from 0.1 to 4 mm) would add to the state of the art the possibility of preparing combined personalised formulations feasible on an industrial scale. A direct consequence of the introduction of this technology would then be a reduction in the number of different medication formulations that patients have to take daily, coupled with an increase in adherence to treatment.

### DISCLOSURE OF THE INVENTION

The present invention relates to an automated method for preparing personalised medication formulations (capsules) that surprisingly meets both the speed and accuracy requirements for unit dosing. Each capsule contains a specific combination of sub-units with the prescribed active ingredients in the desired strengths, and this combination is prepared individually for each patient or group of patients with identical prescriptions. The sub-units are formulated to meet the conditions of bioequivalence and proportionality, i.e., that the rate of release of each active ingredient from the combination of sub-units corresponds to the rate of release from the original single-component dosage form, and that the total amount of each active ingredient released from the combination of sub-units is proportional to the number of sub-units of that type in the mixture. The sub-unit combination process is designed to be quick, automated, and to provide a precise and verifiable mixture of sub-units according to a specified prescription.

Beyond the state of the art, it allows combining pre-formulated manufactured sub-units (mini-tablets, pellets, granules, liquid marbles) into capsules automatically in a precisely defined number to meet not only the condition of bioequivalence but also enable dose titration. In addition, this method allows the combination of sub-units containing the same active ingredient but with different release profiles, thus surprisingly achieving complex dissolution profiles according to the prescribed requirement.

In contrast to the available state-of-the-art technologies for automatic combining or routine filling of solid pharmaceutical formulations into capsules, said method uses optical control at the output of the dosing device. Due to the rapid feedback and mechanical design of the dosing device, this allows for dosing a precise number of sub-units. The principle of conventional capsule filling, as used in commercially available technologies, is based on gravimetric or volumetric control of the capsule contents. The technology for combining (rather than simply filling) solid dosage forms into capsules typically uses the technique of controlled tapping on a container with a perforated bottom coupled with gravimetric control. None of these techniques is suitable for combining precisely defined numbers of different sub-units of the size under consideration, as they do not achieve the required numerical accuracy per piece.

### Definitions

For the purpose of the present invention, sub-units are particles having a size in the range of from 0.1 to 4 mm, preferably in the range of from 0.5 to 3.5 mm, more preferably in the range of from 0.8 to 3 mm, even more preferably in the range of from 1.2 to 2 mm, and are in the form of mini-tablets, pellets, granules, or liquid marbles. The sub-units contain an active ingredient and possibly pharmaceutical excipients.

Liquid marbles are droplets of active ingredients and possibly excipients encapsulated by nano- or micro-sized particles, prepared by dropping a small amount of liquid onto a layer of non-wettable powder, the particles of which spontaneously disperse at the liquid/air interface. The result is a sphere having some properties of a liquid droplet and, at the same time, of a soft solid. Typically, liquid marbles have size in the range of from 0.1 to 3 mm.

Mini-tablets are solid pressings or compacts made from mixtures of active ingredients and possibly excipients in powder form. They can be obtained by standard pharmaceutical tablet preparation technologies by pressing or compacting powder mixtures of active ingredients and, where appropriate, pharmaceutical excipients. Pharmaceutical excipients for the preparation of mini-tablets include fillers, binders, disintegrants, gliding and anti-adhesive agents, colouring agents, substances capable of modifying the product's behaviour in the digestive tract, stabilisers and corrigents. The pressed or compacted particles may be prepared by direct compression of powder or granulate (granular powder) formed by dry or wet granulation. The mini-tablets take on their characteristic dimensions in the range of from 1 to 4 mm thanks to a suitably selected matrix and appropriately sized punches. The mini-tablet cores thus obtained can then be coated.

Pellets are spherical particles, usually approximately 0.5 to 1 mm in size, containing a mixture of an active ingredient and possibly pharmaceutical excipients. They consist of a core and, where appropriate, a coating. The core may be formed from an edible substrate (placebo) and/or the active ingredient and, where appropriate, pharmaceutical excipients. The pellet cores may be obtained as commercial products or prepared by extrusion and spheronisation. The pellet coating is applied to the cores by spraying the active ingredient and, where appropriate, pharmaceutical excipients.

Granulates (granular powders) are granular aggregates (approximately 0.1 to 0.5 mm in size) of powdered active ingredients and possibly pharmaceutical excipients. They can be prepared by dry or wet granulation.

The above-mentioned solid dosage forms (pellets, mini-tablets or possibly granulates) can be coated in dragee drums or fluidised bed coaters to improve their properties. The resulting coatings bring several advantages to these formulations, including, for example, protection, facilitating product identification, or modifying the release of the active ingredient from the dosage form. Depending on its function, the coating may be formed by a thin film or a thick layer of sugars. The coating thickness ranges from 20 to 1,000 μm.

Coatings can be ‘functional’ and ‘non-functional’ depending on whether or not it affects the kinetics of release of the active ingredient from the dosage form. The non-functional coating does not affect the kinetics but provides properties such as protection from light, protection from moisture, taste masking or ease of swallowing. These coatings are composed of sugars or water-soluble polymers (methylcellulose (MC), hydroxypropylcellulose (HPC), polyvinyl alcohol (PVA), polyvinylpyrrolidone (PVP), hydroxypropyl methylcellulose (HPMC), methyl hydroxyethyl cellulose (MHEC), ethylcellulose (EC) and others). Functional coatings, also known as modified-release coatings, affect the kinetics of the release of the active ingredient from the dosage form and can be further categorised into delayed-release and sustained-release coatings. The delayed release is provided by acid-resistant or enterosolvent coatings, whose solubility depends on the environment's pH. They are composed of methacrylic copolymers (methacrylic acid and ethyl acrylate copolymers, polyvinyl acetate phthalate) and certain cellulose derivatives (e.g. cellacefate, cellulose esters, cellulose acetate phthalate, cellulose acetate trimellitate, hydroxypropyl methylcellulose phthalate, hydroxypropyl methylcellulose acetate succinate, etc.). Sustained release of the active ingredient from the dosage form (usually 6 to 12 hours) is achieved by using coatings made of polymers with poor aqueous solubility (copolymers of methacrylic acid and ethyl acrylate, ethylcellulose (EC), methylcellulose (MC), polyvinyl acetate, cellulose acetate, methacrylate polymers, polyethylene glycol, carnauba wax, beeswax, etc.), where the thickness of the coating layer has a major influence on the release rate.

Other additives that coatings may contain include plasticisers (e.g. polyethylene glycols, glycerol, castor oil, triacetin), colourants or pigments (e.g. titanium dioxide, silicates, insoluble pigments), odour and taste corrigents, and possibly antioxidants.

Due to the small size of the sub-units compared to conventional dosage forms (tablets), there is a more uniform passage through the digestive tract (especially the stomach), which ensures better reproducibility of the medicament concentration in the blood. The high degree of dispersion in the gastrointestinal tract also minimises the risks associated with high local medicament concentrations. Sub-unit formulations also lead to higher adherence in paediatric care. Of the different types of sub-units, mini-tablets offer many advantages, such as relative ease of manufacture in various shapes and sizes, reproducible coating options and a high degree of formulation flexibility.

Capsule means a solid, hollow shell, usually made of gelatine or hydroxypropyl methylcellulose, used as a container for pharmaceutical formulations. It is made up of two hollow parts which fit together. One part (the body of the capsule) is filled with the medicament, and the capsule is then closed with the other part (the cap). The shape of the capsules is preferably cylindrical, ovoidal or round.

A cartridge in the present invention means a rack for placing a plurality of capsules, or bodies thereof, in specific positions to allow the dosing of sub-units into individual capsule bodies. The cartridge may, for example, have a square or rectangular configuration and contain 100 positions for placing the capsules. The square or rectangular cartridge configuration allows for faster handling when placing capsules into the cartridge and uncapping or capping them, compared to holders with a linear or circular (carousel type) configuration.

In the present invention, a process record refers to an archived data file containing information about the process history of individual capsule cartridges in each of the sub-steps of the production line (e.g. information about the size and type of capsules inserted in the sub-step of placing capsules into the cartridge, information about the type and amount of sub-units dosed in each dosing station, etc.).

The object of the present invention is a computer-implemented method for the fully automated production of multiple dosage forms with personalised composition. This method of production is readily scalable to the level of industrial production and comprises the following steps:
(i) a control unit generates an input protocol of a personalised composition of a multiple dosage form, said input protocol containing information about the type and amount of individual sub-units to be dosed, about the type of cartridges to be used for dosing, and about the size, type (for example, gelatine or hydroxypropyl methylcellulose), number and arrangement of capsules (each position in the given cartridge having a specific index (order) by which it can be identified) to be used for the particular personalised dosage form; the input protocol may be generated based on the patient's e-prescription or based on manual or automated input of the qualitative and quantitative composition of the active ingredients and the number of capsules to be produced in a given batch, whereby the control unit converts the amount and type of active ingredients into the amount and type of each sub-unit type to be used. Based on the information on the amount and type of sub-units and the number of capsules required, the control unit evaluates what type and size of capsules to be used for the batch and the type of cartridge to be used for the production of the batch;
(ii) the cartridge of the required type according to the input protocol, marked with a specific machine-readable code (e.g. barcode, QR or RFID), is placed in the automatic transport system; the specific machine-readable code of the cartridge is recorded in the input protocol and serves to uniquely identify the cartridge throughout the whole production process of the batch;
(iii) in at least two dosing stations at least two different types of sub-units are placed (i.e., a different type of sub-unit is placed in each dosing station) of sizes in the range of from 0.1 to 4 mm, preferably in the range of from 0.8 to 2 mm, more preferably in the range of from 1.1 to 1.9 mm, containing a different active ingredient and/or different amounts of the same active ingredient and/or having coatings with different functions and/or different thicknesses (i.e. allowing delayed or sustained release) and possibly containing different pharmaceutical excipients, wherein the different sub-unit types correspond to the sub-unit types generated in the input protocol;
(iv) the automatic transport system transports the cartridge to the means for placing capsules in the cartridge, where a pre-defined number of the given type of capsules is placed in the cartridge according to the input protocol;
(v) the automatic transport system transports the cartridge to the first dosing station, which reads the specific code of the cartridge and, according to the input protocol, doses a pre-defined number of the first sub-units into the capsule bodies placed in the cartridge; the information about the specific code of the cartridge and the position of the capsules being filled is entered in the process record, which is generated in the control unit during the production of the batch;
(vi) the automatic transport system transports the cartridge with capsules to the next dosing station, which reads the specific code of the cartridge and, according to the input protocol, doses a pre-defined number of the next sub-units into the capsule bodies placed in the cartridge; the information about the specific code of the cartridge and the position of the capsules being filled is entered in the process record, which is generated in the control unit during the production of the batch; wherein this step is repeated according to the input protocol until the capsules are filled with all of the pre-defined types of sub-units; the transport of the capsules need not be only sequential, i.e., by moving the capsules under a series of dosing stations arranged in a row that sequentially dose the pellets into the capsules, but the automatic transport system allows for targeted transport of the cartridge with capsules just to the dosing stations containing the required sub-units according to the input protocol;
(vii) the capsules are closed with caps;
   The information from each process step of the above described method of cartridge production and movement in the automatic transport system is stored in the process record in the control unit defined above.
(viii) the quality control of capsules and discarding those that do not match the composition according to the input protocol. This step is based on checking the process record, which is specifically linked to the unique machine-readable code of the specific cartridge with capsules and its individual positions, and contains information from each process step. This is followed by the step of comparing the information from the input protocol with the information from the process record of the particular cartridge with filled capsules. If the information relating to a particular position on the cartridge contained in the input protocol and the process record differs, the capsule at that particular position is removed. The removal of the capsule is carried out by means for removing non-compliant capsules, preferably the means for removing non-compliant capsules is an electrical, mechanical or pneumatic/vacuum manipulator.
(ix) the batch of capsules produced is packaged and labelled with the batch number. Each of steps (viii) and (ix) may be semi-automatic or fully automatic depending on the environment in which the device is intended to be used and on the desired production rate (e.g. hospital pharmacy, industrial manufacturing plant).

In the method according to the present invention, the exact number of sub-units is dosed, not the weight or volume, and furthermore, the quality of the capsules is checked by comparing the process record with the input protocol. The cartridge is labelled with a specific machine-readable code, and the automatic transport system purposely delivers it just to those dosing stations that contain the sub-units intended for the capsules in that particular cartridge. The dosing of the number of sub-units and the targeted movement between dosing stations significantly speeds up the dosing process, as there is no need to wait for the balance to stabilise compared to gravimetric dosing (the dosing mechanisms used for dosing sub-units into capsules in the case of gravimetric dosing would affect the stabilisation of the balance, which controls the number of sub-units dosed, therefore, with gravimetric dosing, one would always have to wait for the balance to stabilise, which would slow down the whole process, or while maintaining the dosing speed, inaccurate dosing of sub-units would inevitably occur). The entire cartridge movement and capsule filling data are recorded in the process record, which can be used to check the quality of the capsules at particular positions in a specific cartridge. Thus, when removing defective capsules, it is only necessary to remove individual capsules from the cartridge and not always the entire batch.

Numerical determination of the number of sub-units leads to a more precise and faster method and its greater robustness to weight fluctuation, caused by the weight dispersion of the pellets. In addition, the numerical dosing mechanism allows possible additional quality control by a different mechanism than the mechanism of dosing (for example, using a balance or a device to identify the chemical composition of the dosed sub-units), which significantly increases the probability of detecting a defective capsule compared to the solution where the dosing and control mechanisms are the same (for example by means of weight). The cost-effectiveness of the method is also a significant advantage over the state of the art, as the cost of the balance and associated electronics far exceeds the cost of the optical gate of the counting mechanism.

In a preferred embodiment, in steps (v) and (vi), additional quality control of the capsules may be further performed using an automatic balance and/or a device for identifying the chemical composition of the dosed sub-units (e.g. an NIR probe), wherein the automatic balance and the device for identifying the chemical composition of the dosed sub-units are feedback-connected to the control unit. The automatic balance records the weight increments as the sub-units are dosed into each capsule and the weight increment information for each capsule is stored in the process record. By analogy, in the case of identification of the chemical composition of dosed sub-units, the chemical composition data are stored in the process record. Thus, during the passage of the cartridge through the dosing section, a dual check (weighing/chemical composition) is carried out in each dosing station in addition to the counting of the sub-units. The results are entered in the process record. Subsequently, by comparing the process record and the input protocol in step (viii), capsules that differ from the data in the input protocol can be detected and subsequently discarded. In one embodiment, step (ii) may be carried out by placing a plurality of cartridge trays at the beginning of the manufacturing process, each tray containing one type of cartridge and each cartridge being marked with its specific machine-readable code (e.g. barcode, QR, more preferably RFID). This specific code is stored in a code database in the control unit and is associated with information relating to the specific cartridge (size of the capsule slots, their positioning and indexing (each slot is assigned a value corresponding to its order)).

In one embodiment in step (iv), means for capsule placement may place capsule bodies or whole empty capsules into the cartridge. In the latter case, the next step is to uncap the capsules placed in the cartridge using capsule opening means so that the sub-units can be dosed in the next step (v). In both cases, the result is a cartridge filled with uncapped capsule bodies. The cartridge is filled with capsules in a particular pattern (e.g. XY grid), and principles known from commercially available semi-automatic and automatic capsule fillers can be used for their subsequent uncapping, if necessary. Preferably, a check (e.g. optical) can subsequently be made to see whether each position in the cartridge is occupied by a capsule, each position in the cartridge having a specific index (order) by which it can be identified. Information on the occupancy/non-occupancy of each capsule position is also stored in the process record.

The specific machine-readable cartridge code is part of the input protocol and contains information about the exact arrangement and size of the individual capsule positions on the given cartridge. This information is important when different types of cartridges are used with different capsule position arrangement and capsule sizes and must be provided to the partial technical means such as the means for placing capsules in the cartridge, the capsule opening means, the dosing stations, the capsule closing means and the means for removing non-compliant capsules from the positions. The partial technical means receive the given information about the size and arrangement of the capsule positions on the cartridge from the machine-readable code. This code is further used to specifically link a production batch of capsules to the process record, which is a summary of the process data in the partial technical means.

The retention of information about individual positions on the cartridge with capsules is particularly advantageous when removing capsules that do not meet quality requirements. Thus, only the non-compliant capsules can be removed without having to remove the entire batch. Furthermore, this information can be used preferably to retrospectively trace potential problems on the production line from a GMP (Good Manufacturing Practice) perspective. The machine-readable cartridge code specifically linking the capsule batch to the process record allows for the retrieval of information about the type of capsules that were placed in the cassette, whether all positions were filled without error when the capsules were automatically placed in the cartridge or information about the specific stations that the cartridge reached, etc. This process record uniquely linked to the machine-readable code represents a protocol on the correct production of capsules and contains information for input, inter-operation and release control.

The code marking of the cartridge can be further advantageously used to control the movement of the cartridge through the automatic transport system so that the cartridge is transported just to the dosing stations containing the sub-units prescribed in the input protocol, thus allowing optimisation of the production of the combined personalised capsules.

The technology is based on precise combining and dosing of a given number of sub-units into capsules. The sub-unit sizes can be in the range of from 0.1 to 4 mm, and the sub-units can be mini-tablets, pellets, granules and liquid marbles, as defined above. Each sub-unit contains the active ingredient and, where appropriate, pharmaceutical excipients to provide the desired dissolution profile of the active ingredient in question. The sub-units may be further modified, for example, contain a coating to modify the dissolution profile (immediate release, gradual release, sustained release, pH-dependent release, etc.). Preferably, the amount of active ingredient in each sub-unit is configured such that by multiplication of the number of sub-units the full range of prescribed strengths of the active ingredient in question can be covered and thus allow for dose titration. Preferably, the sub-units are mini-tablets and/or pellets, more preferably adapted for delayed or sustained release by the presence of the sub-unit coating.

The input protocol of the personalised formulation of the multiple dosage form is based on the patient's prescription/treatment plan issued by the physician. The prescription itself usually contains an identifier to uniquely identify the prescription (prescription number, QR code, etc.), patient's identification data (name and surname, insurance company code, insured person number, permanent address), identification data of the issuing physician (name and surname, department/physician identification number, contact address or telephone number), the name of the relevant medical facility and its registration number, the names of the individual active ingredients and their quantities (e.g. weight), instructions for the pharmacist regarding the preparation and dispensing of the medicine, instructions to the patient about the use of the medicine (dosage regimen, relationship to administration with food, etc.), time information on issuing the prescription and its period of validity.

The actual input protocol is created in the control unit on the basis of the prescribed active ingredients in the prescription and their dosage regimen. Each prescribed medication (with specific regimen of dosage and release of the active ingredient) are converted using software, based on available information on bioequivalence, into the appropriate number of sub-units of the given type, which will then be dosed into capsules. In addition, information on the type of cartridges (cartridge dimensions, location and size of empty capsule positions, cartridge identification-machine-readable code) and capsules (size, type, number) to be used for the specific personalised dosage form must be uploaded to the input protocol.

By combining several different sub-units into one capsule, it is possible to create the desired formulation for a specific patient or a specific group of patients with the same medication according to the input protocol, which, unlike FDC tablets, is entirely flexible in terms of the identity and number of individual active ingredients, as well as their strengths and release profile. The strengths of the individual active ingredients are given by the multiple of contents of the active ingredient in one sub-unit and limited only by the physical volume of the capsule. Thus, with an appropriately chosen composition and combination of sub-units, almost any combination of active ingredients and their strengths and dissolution profiles can be prepared ‘tailored’ for a specific patient, while maintaining the advantages of industrial production (speed, quality, traceability).

Filling the capsules with a pre-defined number of sub-units (steps (v) and (vi)) is a crucial step of the technology presented herein, which distinguishes it from previously used methods of producing solid dosage forms and filling capsules. Before the cartridge is placed in the automated transport system, a unique machine-readable code is assigned to the cartridge and information about the required number of capsules in the cartridge and the specific positions to be occupied by the capsules is entered into the input protocol. The same code is subsequently linked to the process record of the individual steps of manufacturing multiple dosage forms. The cartridge with capsules is transported by the automatic transport system towards the individual dosing stations. Each station doses a pre-defined number of sub-units into the capsules according to the input protocol. This is done by moving the cartridge with capsules below the dosing station outlet (e.g. nozzle) or by moving the dosing station outlet above the cartridge with capsules. Because each dosing station contains different types of sub-units and the cartridge with capsules moves between them, there is no cross-contamination of capsules that would occur if there was only one dosing station and different sub-units were dosed through it sequentially.

Preferably, the speed of dosing sub-units into the capsules can be software-controlled by the control unit from high speeds (approximately 15 sub-units per second) to low speeds (approximately 1.7 sub-units per second). During the change in the relative positions of the outlet of the dosing station and the capsule or during the replacement of cartridges (removal of the cartridge with filled capsules and feeding the cartridge with capsules to be filled), the dosing of the sub-units is turned off. This prevents the loss of sub-units and cross-contamination of adjacent capsules.

More preferably, the dosing into the capsule initially proceeds at a higher speed and slows down as the target number of sub-units is approached to a lower speed that ensures that the pre-defined number of sub-units is dosed into the capsule with single-unit accuracy.

The automatic transport system can be a belt or chain conveyor, a pneumatic conveying system, an electromagnetic conveying system, a robotic manipulator or a combination thereof. Preferably, the conveyor is of a belt or band type.

After the cartridge with capsules leaves the last dosing station, the capsule bodies and caps are automatically joined together to seal the capsules. Closure is preferably accomplished by placing the counterparts with the capsule bodies and caps on top of each other and then mechanically pressing them together.

The object of the present invention is also a robotic device for carrying out the computer-implemented method of manufacturing multiple dosage forms having a personalised composition according to the above-described invention, wherein said robotic device comprises means for performing the steps of said method.

In one embodiment, the robotic device comprises a control unit, an automatic transport system for cartridges, a means for placing capsules in the cartridge, a capsule opening means, at least two dosing stations, a capsule closing means, and a means for removing non-compliant capsules; wherein the automatic transport system for cartridges; the means for placing capsules in the cartridge; the capsule opening means; the dosing stations; the capsule closing means; and the means for removing non-compliant capsules are feedback-connected to the control unit. The control unit is adapted for generating and archiving of the process record from each sub-device (automatic transport system for cartridges; the means for placing capsules in the cartridge; the capsule opening means; dosing station; the capsule closing means and means for removing non-compliant capsules). Each sub-device receives/sends information from/to the control unit, which stores this information in the process record.

The automatic transport system performs the function of a conveyor which may be variously branched, or may include various robotic arms, self-driving carts, etc., and which starts at the beginning of the entire process of manufacturing the multiple dosage forms, i.e. before the means for placing capsules in the cartridge. It serves to transport the cartridge throughout the process between the various sub-devices up to the means for removing non-compliant capsules and, where appropriate, to the means for packaging the batch of capsules produced. In one embodiment, the automatic transport system is selected from the group comprising a belt conveyor, a chain conveyor, a pneumatic conveying system, an electromagnetic conveying system, a robotic manipulator, or a combination thereof. Preferably the automatic transport system is a belt conveyor or a chain conveyor.

In a preferred embodiment, each dosing station is designed to accommodate a different type of sub-units (i.e. sub-units with a different active ingredient or a different concentration of an active ingredient, a different type and/or thickness of the coating, and possibly a different type and/or amount of pharmaceutical excipients). The dosing station is adapted to dose the precise number of sub-units according to the input protocol and includes a cartridge specific code reader so that the dosing information can be paired with the specific cartridge in the process record. Preferably, each dosing station may bear a machine-readable code (e.g. a bar code, QR code, or RFID code) located, for example, on a hopper of the given type of sub-units, to allow control of dosing the correct type of sub-units at said dosing station.

In a preferred embodiment, the dosing stations are grouped into so-called dosing sections, wherein the individual sections contain active ingredients that are often prescribed together. In the dosing stations, the capsules are dosed with sub-units of the desired type and number according to a patient-specific input protocol. A computer program controls the trajectory of each cartridge between the dosing stations to meet the requirement for the combination of sub-units with active ingredients for a given patient while optimising the overall production rate. The number of capsules in a cartridge corresponds to the requirement for the number of doses of the combination of different medicaments for a given patient and period (e.g. 30 to 90 days), plus the number of capsules required for retrospective quality control.

Individual dosing stations ensure that the precise number of sub-units is dosed into each capsule placed in the cartridge according to the input protocol.

In one embodiment, the dosing station includes a sub-unit reservoir, a dosing and counting mechanism, and a positioning device.

The reservoir is adapted to accommodate pre-formulated manufactured sub-units containing the active ingredient. The reservoir may be equipped with a mechanical, optical or electronic lock and key system to prevent the reservoir from being filled with the wrong type of sub-units. The reservoir may be equipped with a system for detecting the amount of sub-units in the reservoir, which alerts the need to refill the reservoir with sub-units if they are running low.

The dosing mechanism is preferably a spiral or linear vibrating dispenser with adjustable vibration frequency. The counting mechanism sends information to the dosing mechanism about the number of sub-units dosed and stops it when the desired number of sub-units is reached. The counting mechanism is preferably an optical beam or a high speed camera detecting the passage of each sub-unit.

The positioning device ensures that the cartridge with capsules moves below the dosing mechanism outlet or the dosing mechanism outlet moves above the cartridge with capsules so that the desired number of sub-units is sequentially dosed into the capsules placed in the cartridge according to the input protocol. The outlet of the dosing mechanism is constructed so that no sub-unit can accidentally fall outside the capsule or skip into an adjacent capsule. The positioning device and the dosing mechanism are controlled by a computer program so that dosing into each capsule is initiated only when the capsule and the dosing mechanism outlet are in the correct mutual positions.

In a preferred embodiment, the dosing station may be further equipped with an automatic balance for additional control of the correct capsule filling, which is independent of the counting mechanism and is also feedback-connected to the control unit. The cartridge containing the capsule bodies is placed on the balance during filling, and the balance records the weight increments as the sub-units are dosed into the individual capsules. The weight gain information for each capsule is archived in the process record, so capsules that would not meet the prescribed quantity of dosed sub-units can be identified and subsequently discarded.

In a preferred embodiment, the dosing station may be equipped with a device for identifying the chemical composition of the dosed sub-units, which is also feedback-connected to the control unit. Preferably, this may be a spectroscopic analysis using a NIR (Near Infrared) or Raman probe. The information on the chemical composition of the sub-units dosed into each capsule is archived in the process record, so capsules that would not meet the prescribed chemical composition of dosed sub-units (content of a particular active ingredient) can be identified and subsequently discarded.

From the data provided online from each dosing station by the balance and/or spectroscopic probes, and stored in the process record, it is possible to determine whether any of the positions in the cartridge contain capsules whose composition does not meet the requirements for the prescribed sub-unit content. After being closed, these capsules are removed using the means for removing non-compliant capsules, preferably using the electrical, mechanical or pneumatic/vacuum manipulator.

Capsules that pass quality control proceed to the packaging and labelling step. The manufactured batch of capsules is preferably marked with identifying data in machine-readable form (e.g. barcode or QR code, RFID tag, etc.) that uniquely links the manufactured batch of capsules to the patient-specific input protocol and process record. The final product is a packaged batch of capsules filled with a patient-specific combination of sub-units according to the specified individual prescription/input protocol.

All components of the robotic device that come into contact with sub-units containing active ingredients are made of materials that meet GMP (Good Manufacturing Practice) requirements, particularly with regard to cleaning and avoiding cross-contamination. As protection against cross-contamination, individual dosing stations may be separated by partitions or housed in separate cubicles, which may be equipped with ventilation. Individual cartridges containing capsule bodies may be enclosed by a cover during transport between dosing stations, the cover being opened only while dosing is in progress.

To optimise the overall production speed, dosing stations are grouped into dosing sections containing sub-units with active ingredients often found in co-prescriptions (they are prescribed together, e.g. cardiovascular medications). In addition, the sub-units with the most frequently prescribed active ingredients can be in more than one dosing station. The system of dosing stations and the automatic transport system that provides cartridge transport between the dosing stations may be linear, circular, tiered, branched, or a combination thereof. The term ‘tiered’ denotes a circular arrangement (or it may be another planar geometric formation) where several of these arrangements are placed vertically, one above the other.

Preferably, this is a combination of segments with a linear layout or so-called assembly islands. In the case of linear segments, each segment always represents one dosing section with sub-units containing active ingredients in co-prescriptions, where the movement of the cartridge with capsules between dosing stations of one section is provided by the belt conveyor or chain conveyor, and the robotic manipulator offers the movement of the cartridge between segments. The assembly island layout production is an analogous system suitable for larger production runs, where individual dosing sections are ‘islands’, i.e. groupings to which the cartridge with capsules is transported, for example, by an autonomous robotic self-driving cart. In both cases (linear segments and ‘island’ production), the movement of the cartridge is controlled by a computer program via the control unit.

In one particular embodiment, the robotic device according to the present invention comprises at least two dosing stations, each having a dosing module for dosing sub-units into individual capsules within the cartridge, a two-axis gantry and an automatic control microbalance coupled to a control unit, which may be, for example, a computer on a chip. The dosing module in this embodiment comprises a vibrating conveyor for transporting the sub-units to a hopper, which provides transport of sub-units to a positionable nozzle. Positioning of the nozzle above each cartridge position or positioning of the cartridge below the nozzle outlet is provided by the two-axis gantry equipped with two stepper motors. When filling the capsules, the nozzle or the cartridge is positioned so that the nozzle outlet is directed above the individual capsules in the cartridge, and each capsule is filled with a pre-defined number of sub-units from the vibrating conveyor according to the input protocol. The dosing of a precisely defined number of sub-units is provided by the dosing mechanism composed of a sorting element, which is located at the end of the vibrating conveyor and ensures that no two sub-units can fall into the hopper at the same time, and an optical sensor, which includes an optical receiver and an optical transmitter, feedback-connected to the control unit. An automatic control microbalance records the total weight gain of the cartridge with capsules during the dosing of sub-units.

In a preferred embodiment, the robotic device according to the present invention comprises at least five dosing stations, more preferably from 15 to 50 dosing stations.

The object of the present invention is also a computer program comprising instructions for the robotic device according to the present invention to perform the steps of the method according to the present invention. The object of the present invention is also a computer-readable medium comprising said computer program.

The multiple dosage form in capsule form, prepared by the method according to the present invention, comprises at least two types of different sub-units having a size ranging from 0.1 to 4 mm, preferably ranging from 0.5 to 3.5 mm, more preferably ranging from 0.8 mm to 3 mm, even more preferably ranging from 1.2 mm to 2 mm, wherein the sub-unit is selected from the group consisting of mini-tablets, pellets, granules and liquid marbles containing an active ingredient and, where applicable, a pharmaceutical excipient.

Preferably, the multiple dosage form comprises coated sub-units, wherein the thickness of the coating is in the range of from 20 to 1,000 μm and the coating material is selected from the group comprising: hydroxypropyl methylcellulose, methylhydroxyethyl cellulose, ethyl cellulose, povidone, or other polymers having a protective function, cellulose esters, cellulose acetate phthalate, cellulose acetate trimellitate, hydroxypropylmethyl cellulose phthalate, hydroxypropylmethyl cellulose acetate succinate, methacrylic acid and ethyl acrylate copolymers, polyvinyl acetate phthalate and other polymers with a similar function, methyl cellulose, polyvinyl acetate, methacrylate polymers, carnauba wax or beeswax, plasticisers (polyethylene glycols, glycerol, castor oil, triacetin), dyes or pigments (titanium dioxide, silicates, insoluble pigments), odour and taste corrigents, and possibly antioxidants.

Most preferably, the multiple dosage form according to the present invention comprises at least two types of different sub-units selected from the group consisting of mini-tablets and pellets, preferably having a size in the range of from 0.8 to 2 mm, wherein the sub-units are adapted for delayed or sustained release of the active ingredient (for example, by the presence of the coating as defined above).

The multiple dosage form according to the present invention may comprise at least one active ingredient selected from the group comprising medications affecting:
cardiovascular system (amlodipine, candesartan, telmisartan, losartan, bisoprolol, metoprolol, carvedilol, atenolol, ramipril, lisinopril, furosemide, lercanidipine, hydrochlorothiazide, indapamide, trimetazidine, digoxin, pentoxifylline)
central nervous system (gabapentin, pregabalin, levodopa, carbidopa, atomoxitin, citalopram, alprazolam, diazepam, clonazepam, zopiclone, zaleplon, zolpidem, carbamazepine, benzotropin, trihexyphenidyl, bromocriptine, valproate, lamotrigine, clobazam)
metabolism (atorvastatin, rosuvastatin, fluvastatin, lovastatin, pravastatin, simvastatin, ezetimibe, fenofibrate, alendronate, metformin, sitagliptin, glimepiride, glipizide, allopurinol)
blood clotting (warfarin, acetylsalicylic acid, clopidogrel)
other systems (tamsulosin, prednisone, levocetirizine, isoniazid, rifampin, ethambutol, pyrazinamide)

In terms of the categorisation of solid dosage forms, capsules prepared by the process described above can be classified as so-called multiple dosage forms, referred to in the literature as either MUDS (Multiple Unit Dosage Systems) or MUPS (Multi Unit Pellet Systems). However, in contrast to FDC (Fixed Dose Combinations), these are variable dosage forms, which can be referred to by the new term ‘PDC’ (Personalized Dose Combinations).

A surprising advantage of the multiple dosage forms produced by the technology described herein is the ability to adjust the release of each active ingredient according to the desired rate profile, allowing for a lower frequency of administration and increased medication efficacy compared to traditional dosage forms. The sub-units are formulated and designed to release the medication at pre-defined rates that depend on the size and structure of the sub-unit. Thus, in addition to immediate release, sub-units and combinations thereof can provide delayed, slow or controlled release. The mentioned advantages cannot be achieved by dosing powder formulations of active substances, the dissolution profile of which is significantly faster due to the larger surface area and, due to the non-uniformity of the particle size and weight of the powder formulations, also difficult to control.

The multiple PDC-type dosage forms described herein are therefore ideal, particularly for patients taking a combination of immediate-, delayed- and slow-release medicaments. For example, suppose a prescription for a given patient contains medications to be taken before and after a meal. In that case, the sub-units can be formulated such that the sub-units containing the active ingredient to be administered before the meal are uncoated and have an immediate release. In contrast, the sub-units containing the active ingredient to be released after the meal are coated to provide a delayed release.

Furthermore, the release of active ingredients from PDCs can be adapted to the individual physiology of each patient (e.g. gastric transit time depending on body structure) by combining sub-units with different release rates, the superposition of which then leads to the desired release profile of the active ingredient in the body. This is not possible with any previously described and standardly manufactured dosage form.

Other advantages of the multiple dosage forms produced by the technology described herein include the ability to combine active ingredients that are not chemically compatible and could not be combined into a single tablet in the traditional manner (e.g. acid and base, hydrate and moisture-sensitive substance, radical source and substance susceptible to oxidative degradation, etc.). Simply mixing such substances in powder form and compressing them into a tablet would result in their mutual degradation. The formulation of the active ingredients into independent sub-units of hundreds of micrometres to sub-millimetres in size, which may also be coated, means that there is no physical contact between the crystals of the individual active ingredients, as is the case, for example, with multilayer tablets.

### BRIEF DESCRIPTION OF DRAWINGS

FIG. 1: A basic schematic representation of the robotic device according to Example 1, comprising a control unit A; a means B for placing capsules in a cartridge; a capsule opening means C; an automatic transport system E for the cartridges; at least two dosing stations D1, D2 located in the dosing section D; a capsule closing means F.

FIG. 2: Dosing station. (A) Overall installation diagram; (B) Detail of the dosing module; (C) Detail of the positioning mechanism with cartridge with capsules and microbalance; (D) Detail of the dosing mechanism. (Legend: 1—dosing module, 2—computer on a chip (Raspberry Pi), 3—two-axis gantry, 4—cartridge with capsules, 5—microbalance, 6—top cover of the dosing module, 7—bottom cover of the dosing module, 8—vibrating dispenser, 9—hopper of the dosing module, 10—hopper reduction, 11—connecting transport hose, 12—positionable nozzle, 13—motor 1 (first axis drive), 14—motor 2 (second axis drive), 15, 16, 17—toothed belts in two axes, 18—sorting element, 19—optical receiver, 20—optical transmitter

FIG. 3: system with branched layout-overall view (left) and top view (right) FIG. 4: System with linear layout-overall view (left) and top view (right)

FIG. 5: System with circular layout-overall view (left) and top view (right)

FIG. 6: Dose titration by multiplying the number of sub-units with (A) rapid release; (B) moderately rapid release; (C) slow release

FIG. 7: Visual comparison of the original dosage form (Concor) with its equivalent in the form of mini-tablets according to Example 4b

FIG. 8: (A) Comparison of the dissolution profiles of Concor 5 (original formulation) and the corresponding amount of mini-tablets in an acidic environment; (B) Comparison of the dissolution profiles of Concor 5 and the corresponding amount of mini-tablets in a neutral environment; (C) Comparison of the dissolution profiles of Concor 10 and the corresponding amount of mini-tablets in an acidic environment; D) Comparison of the dissolution profiles of Concor 10 and the corresponding amount of mini-tablets in a neutral environment, performed according to Example 4b

FIG. 9: Visual comparison of the original dosage form (Lipitor) with its equivalent in the form of mini-tablets according to Example 4c

FIG. 10: (A) Comparison of the dissolution profiles of Lipitor 10 (original formulation) and the corresponding amount of mini-tablets in an acidic environment; (B) Comparison of the dissolution profiles of Lipitor 10 and the corresponding amount of mini-tablets in a neutral environment; (C) Comparison of the dissolution profiles of Lipitor 40 and the corresponding amount of mini-tablets in an acidic environment; (D) Comparison of the dissolution profiles of Lipitor 40 and the corresponding amount of mini-tablets in a neutral environment, performed according to Example 4c

FIG. 11: (A) Dissolution profiles of individual combinations of rapid-release and slow-release mini-tablets; (B) Comparison of measured dissolution profiles of combined formulations with profiles calculated based on the superposition principle

FIG. 12: Personalised dissolution profiles of mini-tablet combinations with different substances in different strengths and with different release types. (A) Sample 1; (B) Sample 2; (C) Sample 3; (D) Sample 4.

FIG. 13: Combination of sub-units representing different shapes, sizes and physical properties (mini-tablets, pellets and liquid marbles) in size 0 gelatine capsules.

FIG. 14: Possibility of influencing the dissolution profile of the active ingredient by choosing the size and structure of the sub-unit. The thickness of the coating influences the time shift of the onset of release (curves 1 to 3), the size of the core influences the rate of release (curves 4 to 5). More complex dissolution profiles can be realized by combining sub-units with different core sizes and coating thicknesses.

FIG. 15: (A) Process diagram for the production of capsules with personalised content. (B) Detail of step (iii), dosing section with a series of dosing stations in a linear layout. (Legend: PC=placing capsules in the cartridge, OC=opening capsules, D=dosing sub-units, CC=closing capsules, QC+RN=quality control and removal of non-compliant capsules, PL=batch packaging and labelling, BPC=batch of personalised capsules, SU=sub-unit)

### EXAMPLES

### Example 1: Robotic Device for Performing a Computer-Implemented Method of Manufacturing Multiple Dosage Forms

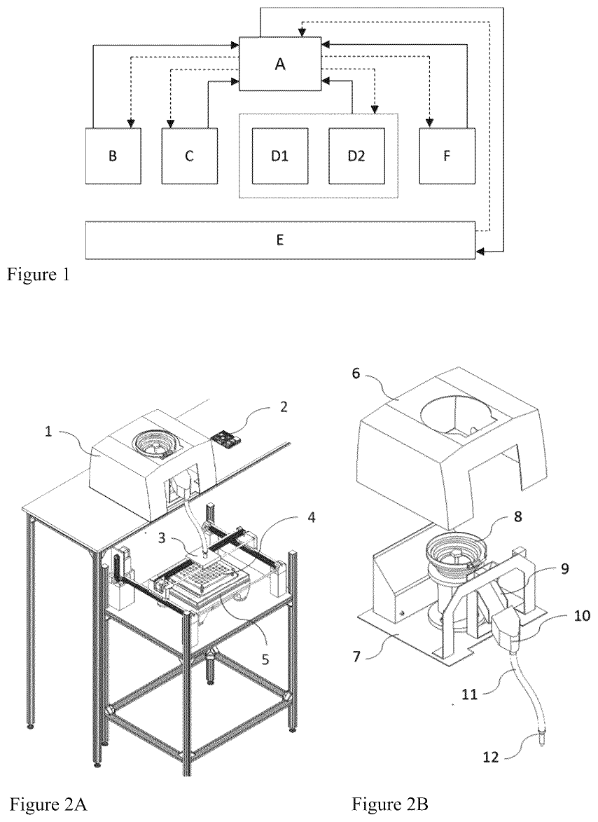

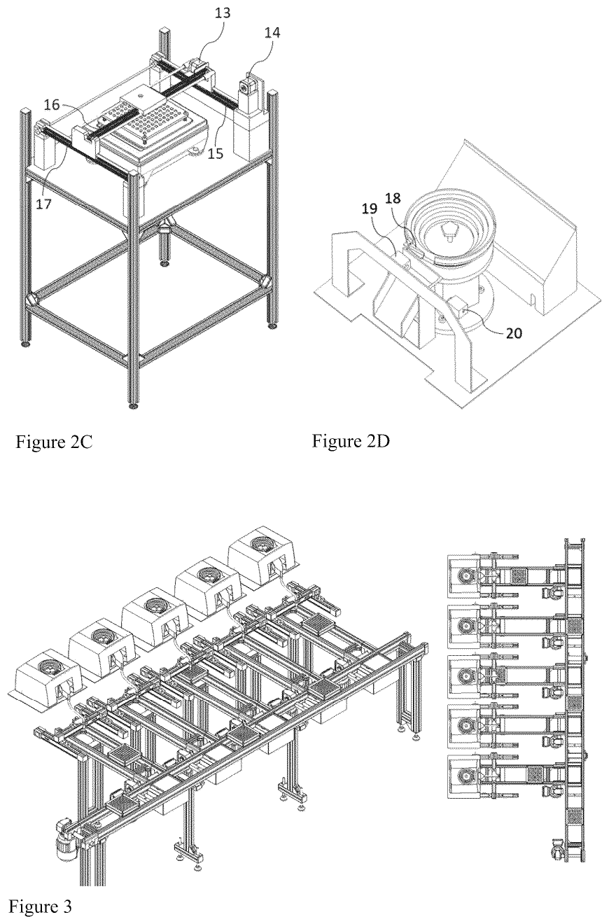

The basic diagram of the device is shown in FIG. 1. The device comprises a control unit A; a means B for placing the capsules in the cartridge; a capsule opening means C; an automatic transport system E for the cartridges with capsules; at least two dosing stations D1, D2 located in the dosing section D; a capsule closing means F, all parts of the device being feedback-connected to the control unit A. The device further comprises a means for removing non-compliant capsules, feedback-connected to the control unit A, which is not illustrated, and a means for packaging and labelling the batch of capsules, which is also not illustrated. A more detailed illustration of the dosing station is shown in FIG. 2.

In this embodiment, the dosing station D1, D2 consists of a dosing module 1 Elmor C3 (Elmor Ltd., Switzerland), a two-axis gantry 3, and of the automatic microbalance 5 (Mettler Toledo WMS404) for the cartridge with capsules 4. The dosing stations contain a cartridge specific code reader. The dosing stations may further comprise a device for identifying the chemical composition of the dosed sub-units, e.g. an IR spectrometer, which is feedback-connected to the control unit A. The control unit A is represented here by a computer-on-a-chip (Raspberry Pi) 2.

The dosing module 1 is composed of a vibrating conveyor 8, which contains sub-units moving upwards and falling into the hopper 9. It is equipped with a plastic reduction 10 (made by 3D printing, material PLA), which is connected to the adjustable nozzle 12 (made by 3D printing, material PLA) using a silicone connecting transport hose 11 (internal diameter 9 mm, length 23 cm). The dosing module 1 is closed from above by the top cover 6, and from below by the bottom cover 7. The two-axis gantry 3 allows, by means of two stepper motors 13 and 14 and three toothed belts 15, 16 and 17 in two axes, precise (accuracy 0.1 mm), automatic positioning of the nozzle 12 above the individual positions of the cartridge 4 with capsules. The nozzle 12 is successively placed above the individual capsules in the cartridge 4, while each capsule is always filled with the required number of sub-units from the vibrating conveyor 8, which is defined in the input protocol. This protocol also contains information about the exact coordinates of the individual positions in the cartridge 4 with capsules. Before starting the dosing itself, information about the type and dimensions of the cartridge 4 is recorded in the input protocol, and the program calibrates the initial position of the nozzle using the end stops. This ensures that when the nozzle is subsequently positioned, the nozzle mouth is always stopped precisely above each individual capsule and the sub-units cannot fall outside the capsule. During dosing of the sub-units into each capsule, the increase in the total weight of the cartridge 4 with capsules is recorded using an automatic microbalance 5, on which the cartridge 4 is placed during dosing. Dosing of a precisely defined number of sub-units is ensured by a dosing mechanism consisting of a sorting element 18 and an optical sensor, which is divided into an optical receiver 19 and an optical transmitter 20. The sorting element 18 is located at the end of the vibrating conveyor 8 and serves to set the width of the end path so that two sub-units do not fit next to each other and thus cannot fall into the hopper 9 at once. Thus, the sub-units fall one after the other and their number is recorded by an optical detector composed of the receiver 19 and the transmitter 20. The automatic microbalance 5, an oscillating device of the vibrating conveyor 8, the motors 13 and 14 and the optical detector (the receiver 19 and the transmitter 20) are feedback-connected to the Raspberry Pi 2, which in this embodiment represents the control unit A.

The speed at which sub-units are dosed from the vibrating conveyor 8 can be controlled by software using the Raspberry Pi 2, ranging from high speeds (approximately 15 sub-units per second) to low speeds (approximately 1.7 sub-units per second). During the movement of the nozzle 12 between the capsules, the dosing is turned off. When dosing the desired number of sub-units into the capsule, the speed is continuously varied to ensure both overall speed and accuracy. Dosing into the capsule initially takes place at a higher speed, and when the target number of sub-units is approached, it slows down to a value that guarantees that the specified number of sub-units is dosed into the capsule with an accuracy of one piece. The dosing speed of the vibrating conveyor 8 is regulated on the basis of information from the optical detector (optical receiver 19 and transmitter 20) about the currently dosed number of sub-units, which enables an automatic reduction of the speed at the moment when the target number of sub-units is approached. At the same time, the weight increments of the cartridge 4 with capsules due to the fall of the dosed sub-units are continuously recorded using the microbalance 5. This record serves as an independent control of the correctness of the dosed number of sub-units into each capsule and can be used to identify and later exclude those capsules that do not meet the specified requirement for the amount of dosed sub-units.

The device shown in FIG. 1 is controlled from a central workstation by a SCADA (Supervisory Control and Data Acquisition) system, which is software that monitors the partial technical means within the production line. Automatic control of individual technical means is provided by PLC (Programmable Logic Controller). This is an industrial computer that controls each individual piece of technical equipment (e.g. the dosing section).

The PLC sends and receives information from/to individual sensors and actuators (motors, valves, etc.) and the SCADA sends and receives information from/to individual PLCs. All PLCs within the production line form a DCS (Distributed Control System) network, which together with the SCADA system constitutes the control unit A. The SCADA collects and stores information from the DCS system (individual PLCs), which is further processed within the classical MIS/MES (Manufacturing Information System/Manufacturing Execution System) systems used to collect, archive, visualise and evaluate data, or to evaluate the individual statuses of devices and create automatic control interventions (in the case of MES systems). The SCADA is thus a software superstructure that brings together all the PLCs of the production line, i.e. the PLC controlling the capsule placement, the PLC controlling the capsule opening, etc. Several SCADA systems can be present within one factory, which then send information to the MIS/MES system, which in turn forms the SCADA superstructure and serves managers to collect, archive, visualise and evaluate data. Compared to MIS, the MES system also includes replacing managerial work with a robot, an algorithm makes managerial decisions.

The process flow diagram for the production of the personalised capsules is shown in FIG. 15. An example of the decision-making algorithm taking place in the DCS system of the production line according to FIG. 15 is as follows:
1) Load the list of medicinal substances in given strengths (from the patient's e-prescription, or other method of input)
2) Check that the list contains all necessary information
   YES—start the capsule batch production process and proceed to step 3
   NO—show error message and return to step 1
3) Convert the list of specified medicinal substances to the corresponding sub-unit types and their equivalent number and proceed to step 4
4) Evaluate the size of capsule to be used for the batch based on the total number and types of sub-units and proceed to step 5
5) Based on the input (patient's e-prescription or other method of input), evaluate the total number of capsules within the batch to be produced (including a margin for retrospective quality analysis and possible removal of non-compliant capsules) and proceed to step 6
6) From the total quantity and type of the given capsules, determine which type of cartridge will be used for the given batch and which positions will be occupied on the cartridge (according to the increasing index). Lock the other positions for further processing steps and proceed to step 7
7) Check that there is a matching cartridge for capsules in the empty cartridge station
   YES—proceed to step 8
   NO—show error message and stop batch production
8) Place the appropriate type of cartridge at the beginning of the automatic transport system E and proceed to step 9
9) Transport the cartridge to the step of automatically filling the capsules into the cartridge and proceed to step 10
10) Check that the cartridge is in the station for filling the capsules into the cartridge
   YES—proceed to step 11
   NO—repeat step 10
11) Place the given capsule type in the specified positions on the cartridge based on the information from step 6 and proceed to step 12
12) Check that all capsule positions have been filled with the correct type of capsules
   YES—proceed to step 13
   NO (wrong capsule type)—show error message, lock all positions for filling steps, mark them as FAILURE and proceed to step 13
   NO (vacant positions)—show error message, record vacant positions, lock them for filling steps and proceed to step 13
13) Transport the cartridge to the step of automatically opening the capsules and proceed to step 14
14) Check that the cartridge is in the station for opening capsules
   YES—proceed to step 15
   NO—repeat step 14
15) Open the capsules and proceed to step 16
16) Check that all capsules in the cartridge have been opened
   YES—proceed to step 17
   NO—show error message, record positions with not-opened capsules, mark them as FAILURE, lock them for filling steps and proceed to step 17
17) Transport the cartridge to the appropriate dosing station and proceed to step 18
18) Check that the cartridge is in the dosing station containing the sub-units prescribed in the input protocol
   YES—proceed to step 19
   NO—go back to step 17
19) Fill the first unlocked position with the capsule body with the prescribed amount of sub-units and proceed to step 20
20) Check for a specific position that the prescribed number of sub-units has been filled
   YES—mark the capsule in the given position as SATISFACTORY, lock the position for filling at the current station and proceed to step 21
   NO—mark the capsule in the given position as FAILURE, lock the position for filling at the current station and proceed to step 21
21) Check that all positions are locked on the cartridge at the current dosing station
   YES—proceed to step 22
   NO—go back to step 19
22) Check that the cartridge contains all prescribed sub-unit types according to the input protocol
   YES—proceed to step 23
   NO—go back to step 16
23) Transport the cartridge to the capsule closing station and proceed to step 24
24) Check that the cartridge is in the capsule closing station
   YES—proceed to step 25
   NO—repeat step 24
25) Close the capsules in the positions marked as SATISFACTORY and FAILURE and proceed to step 26
26) Check that all the capsules in the positions marked as SATISFACTORY and FAILURE have been closed
   YES—proceed to step 27
   NO—mark positions that have not been closed as FAILURE and proceed to step 27
27) Transport the cartridge to the step of removing the capsules marked as FAILURE and proceed to step 28
28) Check that the cartridge is in the station for removing capsules marked as FAILURE
   YES—proceed to step 29
   NO—repeat step 28
29) Remove capsules marked as FAILURE and proceed to step 30
30) Check that all capsules marked as FAILURE have been removed
   YES—proceed to step 31
   NO—go back to step 29
31) Transport the cartridge to the capsule packaging step and proceed to step 32
32) Check that the cartridge is in the capsule packaging station
   YES—proceed to step 33
   NO—repeat step 32
33) Move the capsules from the cartridge to the package and proceed to step 34
34) Check that there are no capsules left in the cartridge
   YES—proceed to step 35
   NO—show error message and go back to step 33
35) Label the package of the batch and proceed to step 36
36) Check that the packaging of the batch has been marked
   YES—proceed to step 37
   NO—go back to step 35
37) Close the production process for the given batch and save the process record including all error messages into the database

### Example 2: Accuracy and Speed of Dosing Sub-Units According to the Specified Prescription

The following tables (Table 1 and Table 2) summarise the characteristics of filling accuracy and the time required to fill 10 capsules with the prescribed number of sub-units in the dosing station D1 (or D2) according to Example 1. Filling correctness characteristics were evaluated for two types of sub-units: mini-tablets (biconvex, 2 mm diameter) and pellets (spherical, 1 mm diameter). The first column shows the prescribed number of sub-units filled into each capsule of size 0. In the second column, the filling speed is expressed in arbitrary units (Elmor software), which are in the range 1 to 10 and express the vibration frequency of the conveyor. In the case of the mini-tablets, the last four pieces were always dosed at a lower rate (speed 1) to avoid over-dosing more sub-units than specified. In the case of pellets, the last six pieces were always dosed at this lower speed. The third column shows the time taken to fill 10 capsules with the prescribed number of sub-units in the format [mm: ss]. In the last column is the error rate parameter, which is expressed as the number of capsules that did not contain the required number of sub-units in proportion to the total number of capsules filled. In the vast majority of cases, the non-compliant capsules differed from the specified number by one sub-unit. In rare cases, there were also capsules that differed by two sub-units. These cases occurred with pellets at higher vibration speeds (speed 6). The number of sub-units by which the failed capsule differs from the input specification has no effect on the defined error rate parameter.

TABLE 1
Dosing accuracy and speed for 2 mm diameter biconvex mini-tablets
Mini-tablets (2 mm, biconvex)

| | | | Error rate |
|---|---|---|---|
| Number of pieces | Speed | Time | (10 repeats) |
| 10 | 2 | [01:36] | 0/10 |
| 10 | 4 | [01:15] | 0/10 |
| 10 | 6 | [01:37] | 0/10 |
| 20 | 2 | [01:55] | 0/10 |
| 20 | 4 | [01:34] | 0/10 |
| 20 | 6 | [02:02] | 0/10 |
| 30 | 2 | [02:06] | 0/10 |
| 30 | 4 | [01:46] | 0/10 |
| 30 | 6 | [02:40] | 0/10 |

TABLE 2
Dosing accuracy and speed for 1 mm diameter spherical pellets
Pellets (1 mm, spherical)

| | | | Error rate |
|---|---|---|---|
| Number of pieces | Speed | Time | (10 repeats) |
| 10 | 2 | [01:30] | 0/10 |
| 10 | 4 | [01:21] | 1/10 |
| 10 | 6 | [01:32] | 4/10 |
| 20 | 2 | [01:52] | 0/10 |
| 20 | 4 | [01:33] | 0/10 |
| 20 | 6 | [01:40] | 5/10 |
| 30 | 2 | [02:14] | 0/10 |
| 30 | 4 | [01:55] | 1/10 |
| 30 | 6 | [02:09] | 3/10 |

To compare with the speed of capsule filling by a human worker, the average time to fill 10 capsules with both types of sub-units was measured. The average time required to fill 10 capsules with 10 mini-tablets while ensuring a 0/10 error rate parameter was approximately 4 min. The average time required to fill 10 capsules with 20 mini-tablets with an error rate of 0/10 was approximately 8 min. Considering the filling method (manual picking of sub-units from the storage container followed by transport into the capsules using protective gloves) and the measured average filling times, in the case of filling 10 capsules, a linear dependence between the number of dosed sub-units and the required filling time can be assumed (see Table 3). For 1 mm pellets, the average time required to fill 10 capsules with 10 pellets while ensuring a 0/10 error rate parameter was higher (approximately 9 min) due to the difficulty in handling smaller particles. However, it must be considered that as the number of capsules to be filled increases, the speed and accuracy of the human worker is likely to decrease. The measured times therefore represent values that the worker would achieve with a very high level of concentration.

TABLE 3
Dosing accuracy and speed of a human
worker for mini-tablets and pellets

| | Number of sub-units | | Error rate |
|---|---|---|---|
| Sub-unit type | in the capsule | Time | (10 repeats) |
| mini-tablet (2 mm) | 10 | [04:00] | 0/10 |
| mini-tablet (2 mm) | 20 | [08:00] | 0/10 |
| mini-tablet (2 mm) | 30 | [12:00] | 0/10 |
| pellet (1 mm) | 10 | [09:00] | 0/10 |
| pellet (1 mm) | 20 | [18:00] | 0/10 |
| pellet (1 mm) | 30 | [27:00] | 0/10 |

### Example 3a: Automatic Transport System E-Transport and Dosing System with a Branched Layout

The branched transport and dosing system (FIG. 3) consists of a pallet conveyor with transverse segments at the end of which is the dosing station described in Example 1. The pallets are equipped with an RFID chip containing information about the number and type of sub-units to be dosed into each capsule. Based on this information, the movement of the pallet (with the cartridge containing the capsules) along the conveyor and the dosing of the sub-units at each dosing station is controlled.

### Example 3b: Automatic Transport System E-Transport and Dosing System with a Linear Layout, which is Analogous to Example 3a without Transverse Segments

This embodiment (FIG. 4) represents a less flexible system than system in Example 3a in terms of production optimisation, but requires lower investment costs.

### Example 3c: Automatic Transport System E-Transport and Dosing System with a Circular Layout

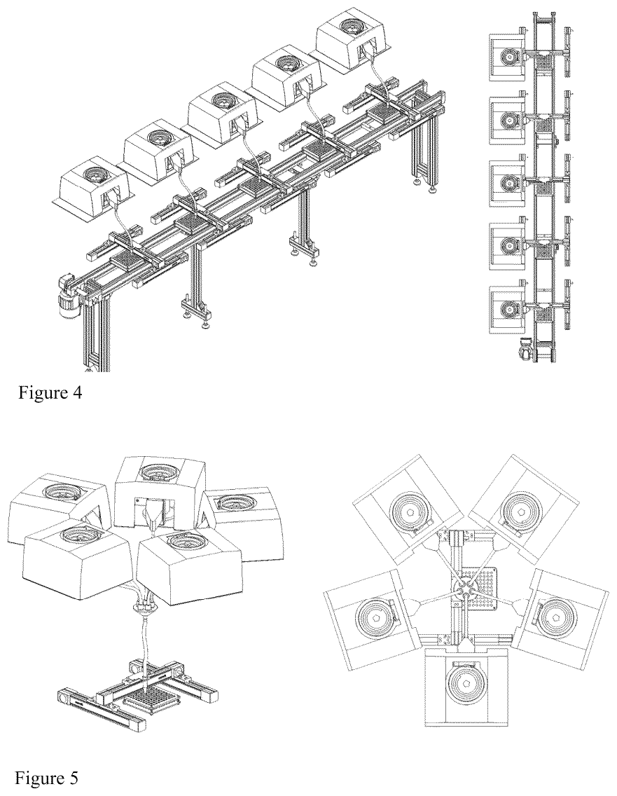

This system (FIG. 5) does not require a pallet conveying device. All vibrating modules dose sub-units in parallel into the same hopper, which is connected by a transport hose to a nozzle positionable above the cartridge with capsules. This layout is suitable for smaller numbers of modules, and the parts in which different types of sub-units occur simultaneously must be cleaned or replaced to avoid cross-contamination.

### Example 4a: Dose Titration by Multiplying the Number of Sub-Units

Dose titration is arranged by multiplying the number of uniform sub-units in the hard gelatine capsules. The possibility of dose titration is demonstrated by three types of sub-units which differ in the rate of release of the active ingredient. All sub-units are represented here by mini-tablets of 1.5 mm diameter and 4 mg weight, prepared by direct compression of the tablet material. For simplicity, instead of the active ingredient, a dye is added to each sub-unit, whose release rate is modified by the addition of different pharmaceutical excipients to each type of mini-tablet and by the compression force of the tabletting process.

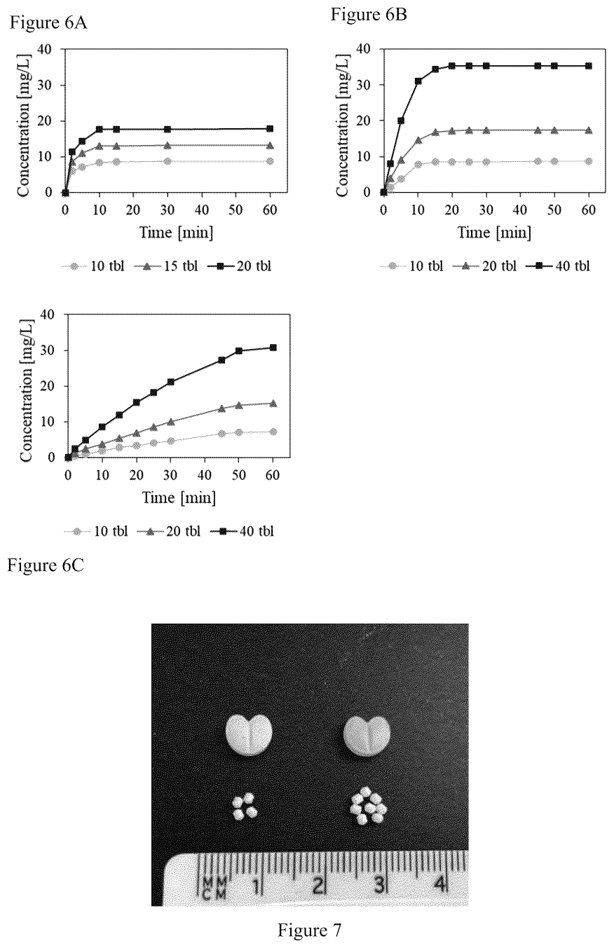

An overview of the mini-tablet types, their strength and the number of pieces tested are summarised in Table 4. The possibility to perform dose titration by multiplication of the number of sub-units, and thus indirectly the content uniformity, was tested using in vitro dissolution assays in a USP 2 apparatus (75 rpm; laboratory temperature, 900 mL water). The measured dissolution profiles for each type and amount of mini-tablets are shown in FIG. 6. For each dissolution experiment, the theoretical dye concentration was calculated when the respective number of mini-tablets was completely dissolved, assuming content uniformity, i.e. each mini-tablet contains exactly 0.8 mg of dye. These theoretical concentrations were then compared with concentrations measured in 60 min of dissolution experiments, i.e. after complete dissolution of the mini-tablets. The relative deviation was then determined as the difference between the two concentrations (theoretical and experimental) relative to the nominal value of the theoretical concentration. This value should not exceed 5 percent according to current regulations in the case of pharmaceuticals. The values of the two concentrations together with the relative deviation are shown in Table 4.

TABLE 4
Overview of sub-units used to test the possibility of dose titration

| | Mini-tablet | Quantity of | Theoretical | | Measured | | Relative |
|---|---|---|---|---|---|---|---|
| Tablet type | strength | mini-tablets | concentration | | concentration | | deviation |
| Fast | 0.8 mg/mini tab. | 10 pcs | 8.9 | mg/L* | 8.7 | mg/L | 2.2% |
| release | | 15 pcs | 13.3 | mg/L | 13.3 | mg/L | 0.0% |
| | | 20 pcs | 17.8 | mg/L | 17.8 | mg/L | 0.0% |
| Moderately | 0.8 mg/mini tab. | 10 pcs | 8.9 | mg/L | 8.8 | mg/L | 1.1% |
| rapid release | | 20 pcs | 17.8 | mg/L | 17.4 | mg/L | 2.2% |
| | | 40 pcs | 35.6 | mg/L | 35.3 | mg/L | 0.8% |
| Slow | 0.8 mg/mini tab. | 10 pcs | 8.9 | mg/L | 8.8 | mg/L | 1.1% |
| release | | 20 pcs | 17.8 | mg/L | 17.7 | mg/L | 0.6% |
| | | 40 pcs | 35.6 | mg/L | 35.4 | mg/L | 0.6% |

*Calculated as the number of mini-tablets (10) multiplied by the strength (0.8) and divided by the volume of the dissolution medium (0.9 L).

The results show that the sub-unit multiples for all types of mini-tablets with different release kinetics proportionally allow dose titration with a relative deviation of less than 5 percent. The kinetics of medicament (dye) release is maintained independent of the number of sub-units and the release from the sub-unit multiples is additive in nature. In addition, for each type of mini-tablet, the same proportion of incorporated dye is always released at a given time, allowing prediction of dissolution profiles at arbitrary titration doses. It is therefore possible to use the principle of superposition of dissolution profiles for the sub-units manufactured and tested.

### Example 4b: Substitution of the Original Medicinal Product by an Appropriate Number of Sub-Units in Terms of Dose Titration and Achievement of Equivalent Dissolution Kinetics-Bisoprolol

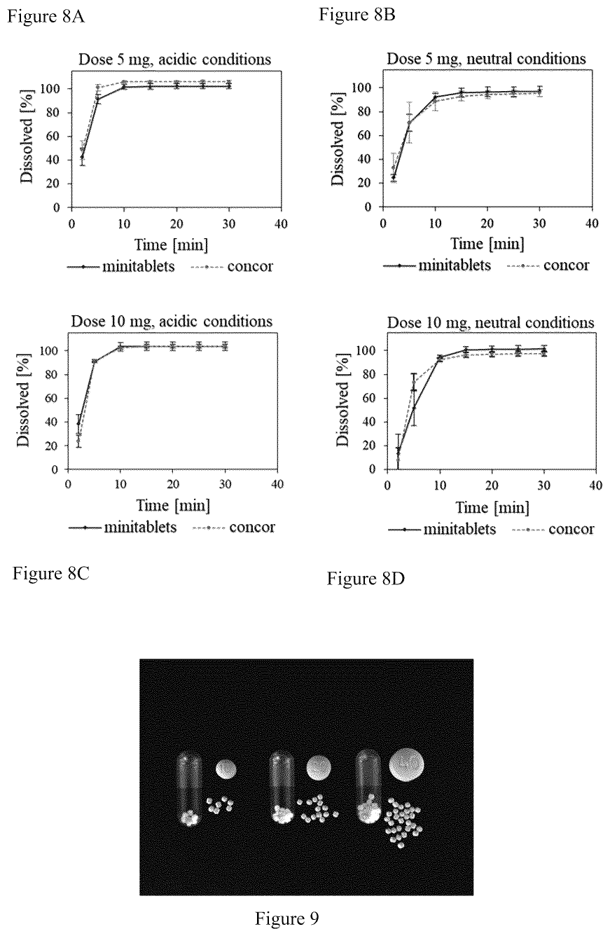

In this embodiment, the active ingredient bisoprolol fumarate belonging to the BCS I group was selected, i.e. the active ingredient with good solubility and absorption. Bisoprolol is commonly prescribed in the treatment of cardiovascular diseases under the trade name Concor in 5 and 10 mg strengths. As an equivalent pharmaceutical formulation, mini-tablets of 1.5 mm diameter and bisoprolol content of 41.25 percent (w/w) were prepared on a tabletting machine by direct compression. The weight of each mini-tablet was 3 mg and the active ingredient content was 1.25 mg. Thus, the equivalent of Concor 5 mg was 4 mini-tablets and Concor 10 mg was 8 mini-tablets. A visual comparison of the different formulations is shown in FIG. 7. In addition to the active ingredient, the formulated mini-tablets also contained microcrystalline cellulose (filler), crospovidone (disintegrant), magnesium stearate (gliding agent) and aerosil. The equivalence of the release kinetics of the active ingredient from a given formulation and the accuracy of dose titration by multiplying the number of sub-units were verified by in vitro dissolution tests in a USP 2 apparatus (50 rpm, 37° C., peak dissolution vessels, 30 min) in both acidic (10 mM HCl solution with pH 2.0) and neutral environments (72.4 mM phosphate buffer with pH 6.8). FIG. 8 compares the dissolution profiles of each Concor strength with the profiles of the corresponding number of mini-tablets. Each of the profiles was evaluated as the average of triplicate and the relative error of the measurements was also evaluated. From the comparison of the profiles, it can be clearly seen that the kinetics of bisoprolol release from Concor and from the corresponding number of mini-tablets are virtually identical. Thus, in the case of bisoprolol, by appropriate formulation of the mini-tablets, equivalent dissolution profiles of the original product and its counterpart in a given number of mini-tablets were achieved. The possibility of dose titration by multiplying the number of mini-tablets in this case is shown in Table 5. The left column of the table contains a description of the formulation and the environment in which the dissolution method was performed. In the next column is the total amount of bisoprolol released from the formulation in question. This is followed by the standard deviation of the measurement and the relative standard deviation, which is related to the theoretical prescribed dose, i.e. 5 mg in the case of Concor 5 (or 4 mini-tablets) and 10 mg in the case of Concor 10 (or 8 mini-tablets). This relative deviation should not exceed 5 percent.

TABLE 5
Demonstration of the possibility of dose
titration in the case of bisoprolol

| | m (Bisoprolol) | | |
|---|---|---|---|
| Type | [mg] | SD [mg] | RSD [%] |
| Concor 5, acidic environment | 5.30 | 0.06 | 6.0 |
| 4 pcs mini-tablets; acidic | 5.08 | 0.12 | 1.6 |
| environment | | | |
| Concor 5, neutral environment | 4.88 | 0.07 | 2.4 |
| 4 pcs mini-tablets; neutral | 5.19 | 0.29 | 3.8 |
| environment | | | |
| Concor 10, acidic environment | 10.24 | 0.38 | 2.4 |
| 8 pcs mini-tablets; acidic | 10.36 | 0.49 | 3.6 |
| environment | | | |
| Concor 10, neutral environment | 9.36 | 0.20 | 6.4 |
| 8 pcs mini-tablets; neutral | 10.08 | 0.36 | 0.8 |
| environment | | | |

### Example 4c: Substitution of the Original Medicinal Product by an Appropriate Number of Sub-Units in Terms of Dose Titration and Achievement of Equivalent Dissolution Kinetics-Atorvastatin

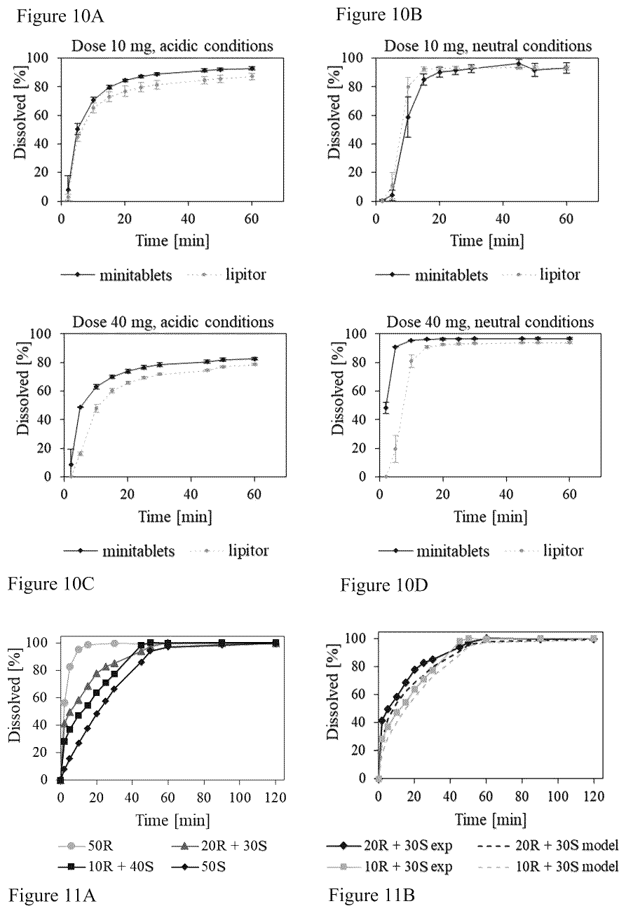

In this embodiment, the active ingredient atorvastatin calcium trihydrate belonging to the BCS II group was selected, i.e. the active ingredient with low solubility and good absorption. Atorvastatin is commonly prescribed in the treatment of cardiovascular diseases under the trade name Lipitor in 10, 20, 40 and 80 mg strengths. As an equivalent pharmaceutical formulation, mini-tablets of 1.5 mm diameter and atorvastatin content of 49.80 percent (w/w) were prepared on a tabletting machine by granulate compression. The weight of each mini-tablet was 2.87 mg and the active ingredient content was 1.43 mg. Thus, the equivalent of Lipitor 10 mg represented 7 mini-tablets and Lipitor 40 mg represented 28 mini-tablets. A visual comparison of the different formulations is shown in FIG. 9. In addition to the active ingredient, the formulated mini-tablets also contained PVP (binder), croscarmellose (disintegrant), sodium carbonate (pH modifier) and magnesium stearate (gliding agent). The equivalence of the release kinetics of the active ingredient from a given formulation and the accuracy of dose titration by multiplying the number of sub-units were verified by in vitro dissolution tests in a USP 2 apparatus (75 rpm, 37° C., classic dissolution vessels, 60 min) in both acidic (10 mM HCl solution with pH 2.0) and neutral environments (72.4 mM phosphate buffer with pH 6.8). FIG. 10 compares the dissolution profiles of each Lipitor strength with the profiles of the corresponding number of mini-tablets. Each of the profiles was evaluated as the average of triplicate and the relative error of the measurements was also evaluated. From the comparison of the profiles, it can be clearly seen that the kinetics of atorvastatin release from Lipitor and from the corresponding number of mini-tablets are virtually identical. Thus, in the case of atorvastatin, by appropriate formulation of the mini-tablets, equivalent dissolution profiles of the original product and its equivalent in a given number of mini-tablets were achieved. The possibility of dose titration by multiplying the number of mini-tablets in this case is shown in Table 6. The left column of the table contains a description of the formulation and the environment in which the dissolution method was performed. In the next column is the total amount of atorvastatin released from the formulation in question. This is followed by the standard deviation of the measurement and the relative standard deviation, which is related to the theoretical prescribed dose, i.e. 10 mg in the case of Lipitor 10 (or 7 mini-tablets) and 40 mg in the case of Lipitor 40 (or 28 mini-tablets). This relative deviation should not exceed 5 percent.

TABLE 6
Demonstration of the possibility of dose
titration in the case of atorvastatin

| | m (Lipitor) | | |
|---|---|---|---|
| Type | [mg] | SD [mg] | RSD [%] |
| Lipitor 10, acidic environment | 8.71 | 0.21 | 2.4 |
| 7 pcs mini-tablets; acidic | 9.28 | 0.11 | 1.2 |
| environment | | | |
| Lipitor 10, neutral environment | 9.31 | 0.13 | 1.4 |
| 7 pcs mini-tablets; neutral | 9.68 | 0.37 | 0.4 |
| environment | | | |
| Lipitor 40, acidic environment | 31.46 | 0.24 | 0.8 |
| 28 pcs mini-tablets; acidic | 33.06 | 0.31 | 1.3 |
| environment | | | |
| Lipitor 40, neutral environment | 37.55 | 0.22 | 0.6 |
| 28 pcs mini-tablets; neutral | 38.67 | 0.18 | 0.7 |
| environment | | | |

### Example 5: Realisation of Personalised Dissolution Profiles by Combining Sub-Units with Different Release Kinetics

In this embodiment, rapid- and slow-release sub-units (1.5 mm diameter mini-tablets) are combined in different ratios. The ratios that represent 100 percent of the rapid- and slow-release sub-units respectively represent the limiting dissolution profiles that can be achieved using the sub-units in question. By selecting an appropriate ratio of rapid and slow sub-units, virtually any dissolution profile can subsequently be achieved, which are limited from above by the dissolution profile for 100 percent of rapid sub-units and from below by the dissolution profile for 100 percent of slow sub-units.

The release kinetics of each combination of slow and rapid-release mini-tablets were measured by in vitro dissolution tests analogous to those described in Example 4. A total of four experiments were performed in which the rate of dye release from 50 rapid-release mini-tablets (50R), a combination of 20 rapid-release and 30 slow-release mini-tablets (20R+30S), a combination of 10 rapid-release and 40 slow-release mini-tablets (10R+40S) and 50 slow-release mini-tablets (50S) was measured.

The measurement results are shown in FIG. 11A. Next, the measured dissolution profiles for the two combined formulations were compared with the theoretical dissolution profiles for these combinations calculated based on the superposition principle. A comparison of the experimental and model dissolution profiles of these combined formulations is shown in FIG. 11B. From the results it can be seen that for the combined rapid- and slow-release mini-tablet formulations, the release kinetics are maintained and furthermore the additive nature of the dissolution profiles is satisfied. Thus, in the case of combining sub-units with different release rates, the principle of superposition of dissolution profiles can be exploited, which can be advantageously used to predict the release kinetics for different ratios in combined formulations.

### Example 6: Realisation of Personalised Combinations of Different Substances in Different Strengths with Different Release Kinetics

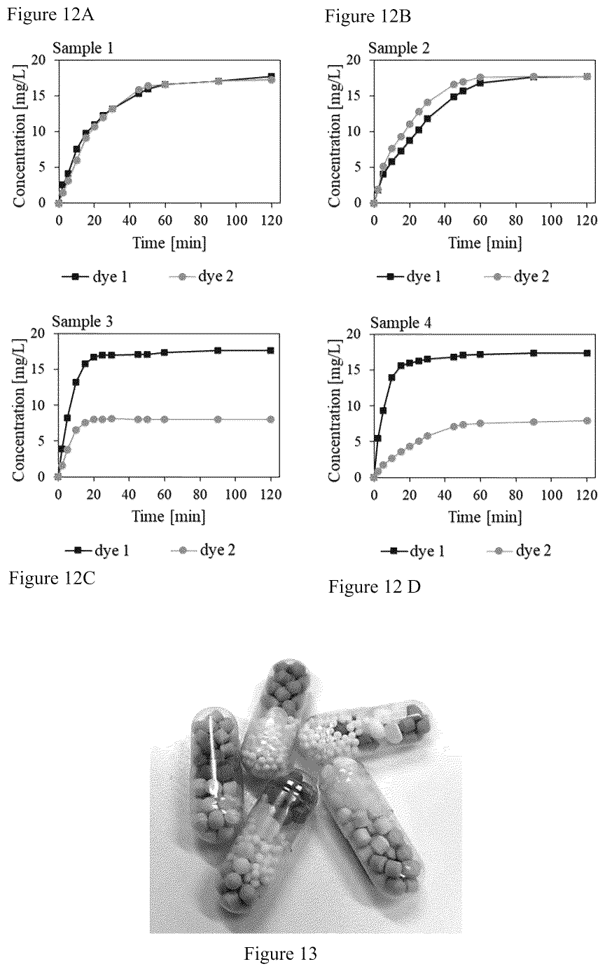

This example is a combination of Examples 4 and 5. For the sub-units tested (mini-tablets of 1.5 mm diameter), two different dyes (representing two medications) are used, different release rates and different number of pieces. The individual combinations for which release kinetics were measured using the same in vitro dissolution assay as in Examples 4 and 5 are summarised in Table 7. The measured dissolution profiles of four selected combinations (marked Samples 1-4) are shown in FIG. 12.

TABLE 7
Summary of mini-tablet combinations used to create personalised
combinations of different substances in different strengths
with different types of release

| | Dye 1 | | Dye 2 | |
|---|---|---|---|---|
| Sample no. | Total dose | Release | Total dose | Release |
| 1 | 16 mg | slow | 16 mg | slow |
| 2 | 16 mg | slow | 12 mg | slow |
| | | | 4 mg | rapid |
| 3 | 16 mg | moderate | 8 mg | moderate |
| 4 | 16 mg | moderate | 8 mg | slow |

The measured results in this embodiment confirm the conclusions drawn from Examples 4 and 5, i.e. that when combining different numbers of sub-units with different substances and different release properties, the pharmaceutical formulations (mini-tablets) used do not interact with each other in terms of release kinetics of individual substances. Thus, using the principle of superposition, the release kinetics of the incorporated substances can be predicted relatively easily and accurately for combined dosage forms such as MUDS capsules. In MUDS capsules, a larger number of medications with almost any release kinetics can be combined at any dose, limited by the physical content of the capsule used.

An example of combined MUDS capsules with sub-units (mini-tablets, pellets and liquid marbles) produced using the apparatus of Example 1 and representing different shapes, sizes and physical properties of the sub-units is shown in FIG. 13.

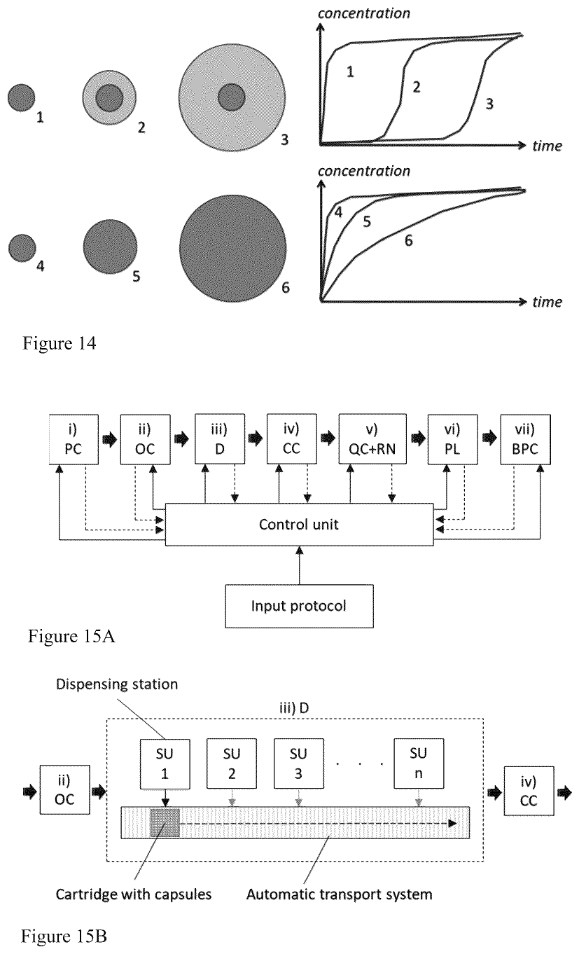

FIG. 14 illustrates the influence of the choice of sub-unit size and structure on the dissolution profile of the active ingredient. The sub-units are formulated and designed to release the medication at pre-defined rates that depend on their size and structure and in addition to immediate release, the sub-units and combinations thereof can provide delayed, sustained, or controlled release.

FIG. 15 then schematically illustrates a process flow diagram of the method of manufacturing capsules with personalised sub-unit content according to the present invention. After the input protocol has been read by the control unit, the capsules are first placed in the cartridge, then opened and successively at least two types of sub-units are dosed therein from the dosing stations. During the dosing process, the quality and accuracy of dosing is checked by means of an optical device. This is followed by closing the capsules, removing any non-compliant ones, packaging and labelling the batch of personalised capsules.

1. A computer-implemented method for preparing multiple dosage forms with personalised composition, comprising the following steps:
(i) a control unit (A) generates an input protocol of a personalised composition of a multiple dosage form, said input protocol containing information about the type and amount of individual sub-units to be dosed, about the type of cartridges to be used for dosing, and about the size, type, number and arrangement of capsules to be used for the particular personalised dosage form;
(ii) a cartridge of the required type according to the input protocol, labelled with a specific machine-readable code, is placed in the automatic transport system (E), wherein the specific machine-readable code of the cartridge is recorded in the input protocol;
(iii) at least two different types of sub-units are placed into at least two dosing stations (DI, D2), the sub-unit having sizes in the range of from 0.1 to 4 mm, and containing a different active ingredient and/or different amounts of the same active ingredient and/or having coatings with different functions and/or different thicknesses and/or possibly containing different pharmaceutical excipients according to the input protocol; wherein the different sub-unit types correspond to the sub-unit types generated in the input protocol;
(iv) an automatic transport system (E) transports the cartridge to the means for placing capsules in the cartridge, wherein a pre-defined number of the given type of capsules is placed in the cartridge according to the input protocol;
(v) the automatic transport system (E) transports the cartridge to the first dosing station (DI), which reads the specific code of the cartridge and, according to the input protocol, doses a predefined number of the first sub-units into the capsule bodies placed in the cartridge;
(vi) the automatic transport system (E) transports the cartridge with capsules to the next dosing station (D2), which reads the specific code of the cartridge and, according to the input protocol, doses a pre-defined number of the next sub-units into the capsule bodies placed in the cartridge; wherein this step is repeated according to the input protocol until the capsules are filled with all of the pre-defined types of sub-units according to the input protocol;
(vii) the capsules are closed with caps;
(viii) the quality control of capsules and discarding of the non-compliant ones using means for removing of non-compliant capsules; and
(ix) step of packaging and labelling with a batch number; wherein the information from each process step ii) to viii) is stored in a process record in the control unit (A); and wherein the quality control of capsules in step viii) is performed by comparing information from the input protocol and from the process record of said cartridge in the control unit (A); wherein if the information relating to a particular position on the cartridge contained in the input protocol and in the process record differs, the capsule at that particular position is removed.

2. The method according to claim 1, wherein in steps (v) and/or (vi), an additional quality control of the capsules is performed using an automatic balance and/or a device for identifying the chemical composition of the dosed sub-units.

3. The method according to claim 1, wherein the sub-unit is selected from a group consisting of mini-tablets, pellets, granulates and liquid marbles.

4. The method according to claim 1, wherein the sub-units comprise a coating for modification of a dissolution profile of said sub-units, and the coating material is selected from the group comprising: hydroxypropyl methylcellulose, methylhydroxyethyl cellulose, ethyl cellulose, povidone, or other polymers having a protective function, cellulose esters, cellulose acetate phthalate, cellulose acetate trimellitate, hydroxypropylmethyl cellulose phthalate, hydroxypropylmethyl cellulose acetate succinate, methacrylic acid and ethyl acrylate copolymers, polyvinyl acetate phthalate and other polymers with a similar function, methyl cellulose, polyvinyl acetate, methacrylate polymers, carnauba wax or beeswax, plasticisers, dyes or pigments, odour and taste corrigents, and possibly antioxidants.

5. A robotic device for carrying out the computer-implemented method for preparing multiple dosage forms with a personalised composition according to claim 1, comprising a means adapted to execute the steps of the method.

6. The robotic device according to claim 5, comprising:
a control unit (A);
a means (B) for placing capsules in the cartridge;
a capsule opening means (C);
an automatic transport system (E) for cartridges, selected from a group comprising a belt conveyor, a chain conveyor, a pneumatic conveying system, an electromagnetic conveying system, a robotic manipulator, or a combination thereof;
at least two dosing stations (DI, D2) for dosing a pre-defined number of sub-units into the capsules, wherein the dosing station (DI, D2) comprises a sub-unit reservoir; a dosing mechanism, preferably the dosing mechanism is a spiral or linear vibrating dispenser with adjustable vibration frequency; a counting mechanism, preferably the counting mechanism is an optical beam or a high speed camera for detecting the passage of each sub-unit; and a positioning device for moving the cartridge with capsules below the dosing mechanism outlet or for moving the dosing mechanism outlet above the cartridge with capsules;
a capsule closing means (F);
a means for removing non-compliant capsules; and
a means for packaging and labelling with a batch number; wherein the means (B) for placing capsules in the cartridge; the capsule opening means (C); the automatic transport system (E) for cartridges; the dosing stations (DI, D2); the capsule closing means (F); the means for removing non-compliant capsules; and the means for packaging and labelling with a batch number are feedback-connected to the control unit (A) and adapted for receiving and transmitting information from and to the control unit (A) for generating of the process record in the control unit (A).

7. The robotic device according to claim 6, wherein the system of dosing stations (DI, D2) and the automatic transport system (E) have linear, branched, circular, tiered, or assembly island layout or a combination thereof.

8. The robotic device according to claim 6, comprising an automatic balance and/or a sensor for chemical analysis, feedback-connected to the control unit (A).

9. A computer program comprising instructions to cause the robotic device according to claim 5 to execute the steps of the computer-implemented method.

10. A computer-readable medium, wherein it has stored thereon the computer program of claim 9.
